(19) United States
(12) Patent Application Publication
(10) Pub. No.: US 2023/0292991 A1
(43) Pub. Date: Sep. 21, 2023

(54) MEDICAL VISUALIZATION AND/OR ILLUMINATION SYSTEM AND METHOD FOR INDICATING NON-VISIBLE ILLUMINATION LIGHT

(71) Applicant: Schölly Fiberoptic GmbH, Denzlingen (DE)

(72) Inventors: Stefan SCHLENKER, Freiburg (DE); Markus GERN, Emmendingen (DE)

(73) Assignee: Schölly Fiberoptic GmbH, Denzlingen (DE)

(21) Appl. No.: 18/118,228

(22) Filed: Mar. 7, 2023

(30) Foreign Application Priority Data
Mar. 16, 2022 (DE) 102022106156.6

(51) Int. Cl. A61B 1/00; A61B 1/06

(52) U.S. Cl. CPC A61B 1/00055; A61B 1/0653; A61B 1/0676; A61B 1/0684

(57) ABSTRACT

To improve the use properties of a visualization and/or illumination system (1), with which an object can be illuminated with non-visible illumination light (2) during a medical intervention, some of the illumination light (2) transported by the system (1) is branched off and guided to a converter (3), which picks up, in particular detects or absorbs, the illumination light (2) and subsequently converts it into an indication signal (4) that is perceivable by humans and outputs this indication signal (4). Using the indication signal (4), a user can thus quickly and easily check whether or not the illumination light (2) is currently being emitted by the system (1).

### CROSS-REFERENCE TO RELATED APPLICATIONS

This application claims priority from German Patent Application No. 10 2022 106 156.6, filed Mar. 16, 2022, which is incorporated by reference herein as if fully set forth.

### TECHNICAL FIELD

The invention relates to a system that can be designed as a visualization system and/or as an illumination system. For example, the system can be implemented in the form of an endoscope, an exoscope or a microscope, with which a respective object can be visualized. However, the system can also simply be designed as an (in particular separate) illumination optical unit. In either case the system is characterized in that it is configured to illuminate an object to be observed with non-visible illumination light in a first wavelength range during a medical intervention.

Moreover, the invention also relates to a method for indicating the presence of non-visible illumination light.

### BACKGROUND

Systems of this type are already used to provide illumination light and excitation light for fluorescence imaging, for example in a laparoscope. In such applications, chromophores such as indocyanine green (ICG) are used, which are excited with the excitation light to spontaneously emit fluorescence light that can then be observed with the system itself or with a separate imaging system. The light source used for the excitation light is frequently an IR laser emitting light in the infrared (IR) wavelength range that is invisible to humans and is potentially dangerous due to its high intensity. However, the systems can for example also emit invisible illumination light in the shortwave UV range, for example if other chromophores that react to such wavelengths are used. Moreover, systems of this type are also already used in order to use invisible illumination light for therapeutic purposes, for example for stimulating nervous tissue.

### SUMMARY

Against this background, it is the object of the invention both to improve the functionality during imaging with systems as described in the introductory part and also to increase the safety for the user. In particular, it is intended to protect said user against dangerous, non-visible light (for example NIR or UV light that is emitted at a high intensity by an LED or a laser).

To achieve this object, one or more features according to the invention are provided in an imaging system and/or system configured for illumination. In particular, to achieve the object in a visualization and/or illumination system of the type mentioned in the introductory part it is thus proposed that the system has a converter that converts the non-visible illumination light into an indication signal that is perceivable by humans. It is preferred here if the converter outputs the indication signal only if the non-visible illumination light is present.

In other words, the invention thus proposes to integrate into the system a converter that converts the illumination light, as soon as it is emitted by the system (for the purposes of illuminating said object), into an indication signal (that is to say an output signal of the system) in a manner such that the presence/the emission of the illumination light can be ascertained or noticed by a person on the basis of the indication signal.

In order to illuminate the object, the system, in particular if it is designed as a visualization system, can have a corresponding illumination optical unit, via which the illumination light can be emitted/transmitted.

Owing to the approach according to the invention, the user is enabled to handle the system safely because said user can always check, on the basis of the perceivable indication signal, whether or not potentially dangerous invisible radiation is being emitted by the system (that is to say for example by an endoscope according to the invention or an illumination apparatus according to the invention). If the indication signal is an indication light, for example, the user can check visually (and thus quickly, easily and reliably) whether the desired invisible light is at all currently being emitted by the system as desired and consequently whether the function of the imaging method/the invisible illumination is ensured. In this way it is thus possible to easily and quickly check the correct function of the system.

The converter can be designed for example as an electrical or electronic component. In that case, the converter can realize, for example, an optomechanical conversion or an optoacoustic conversion. In the first case, the converter will thus convert the illumination light into a mechanical indication signal and output it, for example a vibration signal; in the second case, on the other hand, the converter will convert the illumination light into an acoustic indication signal and output it.

Where a system is mentioned in the following text, this will always be in reference to a visualization and/or illumination system. This is because the invention can be employed, as already mentioned, in endoscopes or other medical imaging systems or, for example, in an illumination system. By way of example, it is possible to advantageously use the invention to convert non-visible illumination light (e.g. laser light in the NIR wavelength range) with the aid of the converter into visible indication light, which indicates to the user the presence and emission of the non-visible illumination light by the system.

Said non-visible illumination light can be, for example, excitation light, in particular from an excitation laser, with the light being used to enable optical excitation, for example for the purpose of fluorescence imaging. The non-visible illumination light, however, can for example also be light that is used for therapy purposes, for example to optically stimulate a specific tissue, which is of interest in particular in neurosurgical applications.

According to the invention, the object can also be achieved by further advantageous embodiments in accordance with the following description and claims.

For example, a preferred configuration proposes that the converter convert at least some of the illumination light into visible indication light. This can preferably be accomplished using a photoactive substance, in particular phosphor. The photoactive substance can then enable or realize optical wavelength conversion of the illumination light into the indication light without any external energy supply. This has the advantage that the indication light can be emitted with a great level of safety and without any additional electronics.

In other words, the indication signal can thus be indication light that is emitted by the converter in order to indicate to a user the presence of the non-visible illumination light. The visible indication light can lie, for example, in a wavelength range from approx. 400 nm to approx. 750 nm.

It is very particularly preferred if said converter is formed by means of a photoactive conversion material. For example, the conversion material can contain phosphor. In a configuration of this type, it is preferred if the conversion material is applied in the form of a (thin) conversion layer on a carrier body; an equivalent alternative can consist in the conversion material being embedded in a transparent carrier body, with it also being possible to combine these two alternatives. Owing to the photoactive conversion material, which can be embodied for example in the form of a conversion layer (preferably having a thickness of less than 0.5 mm), the invisible light can be converted into visible light and thus be rendered perceivable for the user.

The conversion material can thus pick up the invisible radiation of the illumination light and convert it into visible radiation that is re-emitted by the conversion material. Responsible for this can be photoactive substances (such as phosphor) in the conversion material. Said conversion layer, or the conversion material, can thus convert invisible light into visible light on the basis of an effect of photoluminescence, for example on the basis of fluorescence or phosphorescence. Conversion layers of this type have hitherto been known in particular from laser alignment aids that are employed in optical laboratories in order to be able to observe and align invisible laser light.

In physics, the term luminescence subsumes different physical processes, wherein it is characteristic in each of them that a specific emission spectrum of wavelengths is emitted owing to the absorption of a specific absorption spectrum, in particular of one or more excitation wavelengths. Processes on which photoluminescence is based can be, for example, fluorescence or phosphorescence. In very general terms, photoluminescence is thus understood to mean the spontaneous emission of light owing to excitation with excitation light (in the sense of cold-body radiation). There are numerous subtypes of luminescence that are based in particular on chemical or electrical processes or even mechanical processes. In this context, the term photoluminescence is understood to mean luminescence that is brought about by optical excitation radiation. Fluorophores typically do not exhibit any afterglow here, as would be the case with phosphorescence. Phosphorescent substances are accordingly also referred to as luminophores because they can store the light and emit it again at a later point. For the solution to which the present invention is directed, however, very long afterglow should be avoided because otherwise an indication light would still be output when illumination light is no longer present.

A specific configuration proposes that the conversion material contain a photoactive substance that is produced via a sintering method and/or is applied on a ceramic as the carrier body. The ceramic body can for example exhibit a cloudy haze and thus enable some transmission. The scattering of light can also be acceptable for the desired function because the important thing is to indicate visible light to the user. In fact, the scattering of light can be advantageous because it improves the perception of the visible light that is re-emitted by the conversion layer in different spatial directions.

In general, the illumination light can be reflected and/or transmitted by the converter, that is to say in particular by the conversion material. This is because both configurations make it possible for visible indication light to be output to a user.

When converting the non-visible illumination light into the visible indication light, the converter, or the conversion material, can effect for example a conversion from longer wavelengths to shorter wavelengths. In that case, the first wavelength range of the illumination light can lie, for example, in the near infrared (NIR). In addition or alternatively, the converter, or the conversion material, can effect for example a conversion from shorter wavelengths to longer wavelengths when converting the non-visible illumination light into the visible indication light. In that case, the first wavelength range of the illumination light can lie, for example, in the ultraviolet range.

One configuration proposes, especially for endoscopes, that the converter or the conversion material is arranged in a, preferably hermetically sealed, interior space of the system and that the indication light is observable from the outside through a window. Thus, in this case, the indication light is already produced in the interior space and then passes through the window to the outside. The window thus serves as an exit window and enables the indication light to exit from the interior space to the outside. The visible light can thus be observed by the user after it has exited from the exit window and thus makes it possible to draw conclusions relating to the function of the light source that emits the invisible illumination light. The exit window can be designed, for example, as a window that has been soldered in place, for example as a sapphire glass. For this purpose, a gold layer can be applied on a lateral surface of the exit window, with the gold layer enabling the window to be soldered to a metallic housing. A special configuration provides an annular exit window, with the result that the visible indication light re-emitted by the conversion material can be emitted in different spatial directions.

Alternatively or additionally, the conversion can also be such that the converter or the conversion material is applied on the outside of a window and/or is embedded in a/the window. In such a case, the non-visible illumination light can initially pass through the window before it is incident on the conversion material. Thus, in this case, the indication light is only produced outside of the interior space, and for this purpose the illumination light must initially pass through the window to the outside. The window in this case thus serves as a transmission window and thus transmits the invisible illumination light (more specifically a part thereof) to the conversion material.

It is thus also possible for the conversion material, in particular in the form of a conversion layer, to be applied externally on a component of the system. In this case, the invisible illumination light can pass, for example, from a (for example hermetically sealed) cavity/interior space of the system through a window before it is incident on the conversion material. If the conversion material is applied externally in the form of a conversion layer, it is also possible to use a protective layer that is transparent (for the illumination light and also for the indication light) on the conversion layer in order to protect the latter against abrasion.

The converter, that is to say in particular the conversion material, can also be arranged in or on a housing (of the system) that surrounds a light guide, which propagates the invisible illumination light to an illumination optical unit.

Furthermore, it is also (that is to say alternatively or additionally) possible to arrange the converter, that is to say in particular the conversion material, in or on a light-guide cable. The light-guide cable can feed the invisible illumination light for example to an illumination optical unit. In such a configuration, the converter/the conversion material can be arranged in particular in or on a distal termination bushing of the light-guide cable. Such a termination bushing can be used to connect the light-guide cable to an endoscope or to another visualization system. For this purpose, the termination bushing can have, for example, a polished light-guide end face that passes the illumination light to the endoscope. The termination bushing can then have a window as described previously for emitting the visible indication light, in particular an exit window for the indication light (if the conversion material is arranged on the inside) or a transmission window for the non-visible illumination light (if the conversion material is arranged on the outside). Such a window can be designed for example in the form of an (in particular annular) glass sleeve.

Finally, it is also (that is to say alternatively or additionally) possible to arrange the converter, that is to say in particular the conversion material, in or on a catheter, wherein the catheter is configured to introduce the invisible illumination light into a body cavity.

It is preferred in all these cases if the converter, that is to say in particular the conversion material, is arranged such that the converter/conversion material is located extracorporeally when the visualization and/or illumination system is used as intended. This makes it much easier for the user to perceive the indication signal/indication light.

The conversion material can also be arranged on a light-guide cable (as one possible component of the visualization and/or illumination system) on the inside of and/or outside of the light-guide cable. The light-guide cable can have, for example, a flexible light guide that is protected by a cladding, wherein a window as described previously is then provided in the cladding. Further possible configurations of the light guide could be as follows, for example: a liquid-filled light guide or a light guide that is formed from optical glass elements, in particular from rod lenses, or from flexible glass fibres, in particular from fused silica fibres. It is also conceivable that the light guide is an image guide and thus enables structured illumination with the aid of an image-guide bundle.

An illumination system according to the invention could be designed, for example, as a catheter that has a coupling point for coupling a light source or has an internal light source and with which the non-visible illumination light may be introduced into a body cavity. In such a case, it would be useful to arrange the converter, in particular the conversion material, at a point that is located extracorporeally when using the catheter as intended.

According to a further configuration, a light source of the visualization and/or illumination system that emits the illumination light is arranged in an interior space (of the system) and a light guide arranged in the interior space guides the illumination light to the converter/the conversion material. In a configuration of this type, the light source can be arranged in particular in a distal end region of an endoscope (according to the invention). In other words, the light guide can propagate the illumination light in particular in the direction of a proximal end of the endoscope. This can advantageously be configured such that, when the endoscope is used as intended, the indication light can be output extracorporeally. For example, if the light source is arranged in a distal end region of an endoscope (as one example of a visualization system according to the invention), which is typical in particular for chip-in-tip endoscopes, the converter/the conversion material can be located backwards in the proximal direction so that the converter/the conversion material lies outside the body of the patient (extracorporeal) during surgery and can thus be easily observed by the surgeon. In this case, the indication light will not interfere with the imaging in the body; at the same time, it can be easily visibly perceived by the user outside the body of the patient.

One solution for obtaining a visualization and/or illumination system according to the invention consists in the system having an internal light guide forming a branch which branches off some of the illumination light from an optical main path used for the illumination and which guides it to the converter/the conversion material. For example, in such a configuration, individual optical fibres are branched off from a main bundle of optical fibres at the branch. The end faces of these branched-off optical fibres can be polished, and the end faces can be guided, for example, through through-passages in a metal housing, for example an endoscope, from an interior space to the outside. In order to ensure an impermeability of the interior space, the fibre ends can be glued into the housing. Next, the conversion material, preferably in the form of a conversion layer, can be applied on the outer fibre ends in order to thus be able to convert the invisible light transported by the branched-off fibres into the desired visible indication light and to output it.

To achieve the object mentioned in the introductory part, the invention also proposes a method for indicating the presence of a non-visible illumination light. For example, the illumination light can be emitted by a visualization and/or illumination system in order to illuminate an object to be observed during a medical intervention. This system can be designed here in particular as previously described, that is to say it is advantageous if a system according to the invention is used to implement the method. To achieve the object, it is proposed with reference to this method that the non-visible illumination light is converted by a converter, which is preferably part of the visualization and/or illumination system (1), into an indication signal that is perceivable by humans and is emitted.

This method can even be developed further: For example, the converter can pick up some of the illumination light (for example by means of a photodiode) and output an optical indication signal in reaction thereto. For this purpose, the converter can be designed as an optoelectronic converter.

Alternatively or additionally, provision may also be made in the method for the converter to output an acoustic indication signal. In this case, the converter can thus be designed as an optoacoustic converter.

Alternatively or additionally, provision may also be made in the method for the converter to output a mechanical indication signal, in particular a vibration signal. In this case, the converter can thus be designed as an optomechanical converter.

As these numerous configuration examples show, a converter according to the invention can therefore output for example an acoustic, an optical or a mechanical output signal, which in each case indicates the presence of the invisible illumination light and can be perceived by a user. In all of these different configurations of the method it is advantageous for ensuring quick updating of the indication/the output if the converter converts the illumination light (in particular only) if the illumination light is output by the visualization and/or illumination system.

Finally, it is also preferred in the method (in order to increase the reliability of the indication) if the converter performs optical wavelength conversion of the illumination light into an (in particular the previously described) indication light without an external energy supply, preferably based on a photoactive substance such as a phosphor and/or on the basis of photoluminescence. This is because such an action or conversion mechanism is particularly robust, durable and not very susceptible to errors.

### BRIEF DESCRIPTION OF THE DRAWINGS

The invention will now be described in more detail on the basis of exemplary embodiments, but is not restricted to these exemplary embodiments. Further developments of the invention can be obtained from the following description of a preferred exemplary embodiment in conjunction with the general description, the claims and the drawings.

In the following description of various preferred embodiments of the invention, elements that correspond in terms of their function are denoted by corresponding reference numerals, even in the case of a deviating design or shape.

In the figures:

FIG. 1 shows a schematic view of a visualization system according to the invention in the form of an endoscope,

FIG. 2 shows a detailed view of a further visualization system according to the invention in the form of an endoscope,

FIG. 3 shows an illumination system according to the invention in the form of a light-guide cable,

FIG. 4 shows a (spatial) longitudinal section through a visualization system according to the invention which has a hermetically sealed interior space,

FIG. 5 shows a 2D longitudinal section in a different orientation through the visualization system of FIG. 4,

FIG. 6 shows a further possible configuration of the visualization system from FIG. 5 or FIG. 4,

FIG. 7 shows a further visualization system designed in accordance with the invention, with a detail of the housing being shown, and finally

FIG. 8 shows a further longitudinal section through a visualization system that is designed in accordance with the invention.

### DETAILED DESCRIPTION

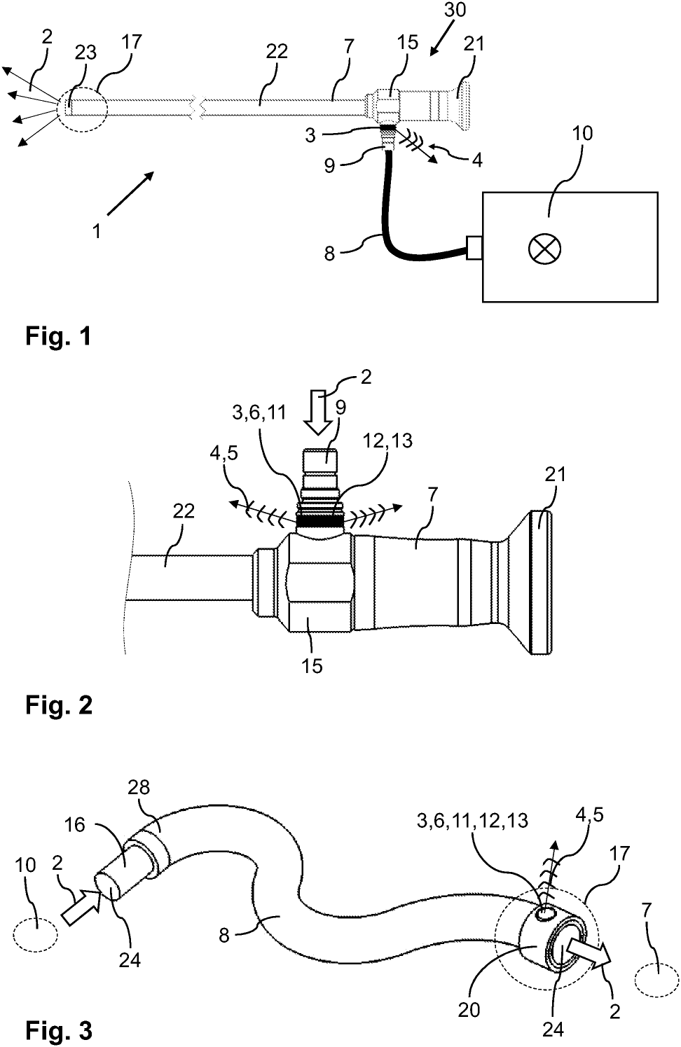

FIG. 1 shows an endoscope 7, which is supplied with invisible infrared illumination light 2 from a light source 10 by means of a light-guide cable 8. Inside the endoscope 7, the invisible illumination light 2 is guided by means of internal light guides (not illustrated) to a distal exit window 23 in the distal end region 17 of the endoscope shaft 22, where the illumination light 2 exits. The infrared illumination light 2 is used here as excitation light to illuminate a tissue region as the object to be observed during a medical intervention. More specifically, the illumination light 2 as excitation light serves to excite fluorophores which were previously introduced into the tissue to spontaneously emit light in order to enable fluorescence imaging of the vessels in this way.

As is shown in FIG. 1, the light-guide cable 8 is coupled to the endoscope 7 via a light-guide connection 9 of the endoscope 7. In the endoscope 7, in the region of the light-guide connection 9, an optoelectronic converter 3 is formed which detects some of the non-visible IR illumination light 2 by means of a photodiode that is specifically provided for this purpose. If the photodiode outputs a corresponding detection signal, the converter 3 outputs an acoustic indication signal 4 that is perceivable by the user of the endoscope. In other words, in this example, the converter 3 is designed as an optoacoustic converter that converts the illumination light 2 (detected by means of the photodiode) into an indication signal 4, specifically the acoustic output signal, that is perceivable by humans.

However, the converter 3 in this case outputs the acoustic indication signal 4 only if the non-visible illumination light 2 is present. If the photodiode does not detect the illumination light 2, no acoustic indication signal 4 is output by the converter 3. It is then possible for the user to check on the basis of the acoustic indication signal 4 whether the light source 10 functions correctly and the non-visible illumination light 2 transmitted by the latter passes via the light-guide cable 8 into the endoscope 7 and is thus emitted by the endoscope 7 in the distal end region 17.

A further possibility would be to design the converter 3 in FIG. 1 in the form of an optomechanical converter, which for example outputs a vibration signal that can be felt by the surgeon while handling the endoscope 7. In all these cases, a method according to the invention is thus implemented, because the converter 3 in each case converts the non-visible illumination light 2 into an indication signal 4 that is perceivable by humans but outputs this indication signal 4 only if non-visible illumination light 2 is in fact being emitted by the respective system 1.

FIG. 2 shows a further example of an endoscope 7 designed in accordance with the invention. This endoscope 7 also has a converter 3 in the region of the light-guide connection 9. This converter 3 also converts some of the illumination light 2 into an indication signal 4; however, in deviation from the example of FIG. 1, the converter 3 does not output an acoustic signal but visible indication light 5. For this purpose, the converter 3 is formed by means of a photoactive conversion material 6 which contains phosphor. The conversion material 6 is formed as a thin conversion layer 11 on the outside of an annular transparent glass window, with the latter acting as a carrier body 12 for the conversion material 6.

Inside the endoscope 7 of FIG. 2, some of the illumination light 2 that was introduced via the light-guide connection 9 is branched off and transmitted from the inside through the window 13 to the outside so that the illumination light 2 is incident on the conversion layer 11 located outside. The conversion layer 11 absorbs the infrared illumination light 2 and re-emits visible indication light 5 in transmission. Since the window 13 is designed to be annular and is irradiated by the illumination light 2 from the inside in multiple directions, the indication light 5 is emitted, as illustrated in FIG. 2, in different spatial directions so that a user can perceive the indication signal 4 from different spatial directions.

As is indicated in FIG. 2, the distal end region 17 of the endoscope 7 can lie in the body’s interior that the surgeon/user of the endoscope 7 inspects via the eyepiece 21. The converter 3 is consequently arranged extracorporeally in this typical use situation, and so the surgeon can perceive the indication light 5 without difficulties. In addition, the indication light 5 also does not pass into the body’s interior, because the visible indication light 5 could interfere with the imaging there. It should be noted at this point that the endoscope 7 may emit not only the non-visible illumination light 2 in the form of excitation light, but also visible illumination light, for example if the endoscope is intended to be used for white light imaging (in particular at the same time as the fluorescence imaging).

FIG. 3 shows a further possible configuration of how a converter 3 according to the invention can be utilized in an illumination system 1 in order to make non-visible illumination light 2 perceivable by a user. The illumination system 1 shown, which can also be used as part of a visualization system 1 according to the invention, is in the form of a light-guide cable 8, which has in its interior a light guide 16 surrounded by a cladding 28. Non-visible illumination light 2 emitted by a light source 10 is coupled into an end face 24 at the proximal end of the light-guide cable (left-hand side of FIG. 3). The internal light guide 16 transports the illumination light 2 into a distal end region 17 of the light-guide cable 8 (right-hand side of FIG. 3). Formed at the distal end of the light-guide cable 8 is a metallic termination bushing 20, which - similar to the examples of FIG. 1 and FIG. 2 - serves to mechanically connect/couple the light-guide cable 8 to a light-guide connection 9 of an endoscope 7. In other words, the illumination light 2 emerges from the end face 24, which can be seen on the very right in FIG. 3, and is then passed on to the endoscope 7.

As is shown in FIG. 3, a circular cutout in which a transparent window 13 has been placed is formed in the termination bushing 20. Unlike the example of FIG. 2, however, in FIG. 3 a conversion layer 11 is applied on the inside on the window 13, which again acts as a carrier body 12. By roughening the light guide 16 and removing the cladding 28 in the region of the window 13, some of the illumination light 2 that is transported by the light guide 16 is coupled out and in this way irradiates the conversion layer 11, which is arranged on the inside. This conversion layer 11, which serves as the converter 3 according to the invention, again converts the non-visible illumination light 2 into visible indication light 5. The conversion layer 11 also emits the indication light 5 at least partially in the direction illustrated as an arrow in FIG. 3, as a result of which some of the indication light 5 is transmitted from the inside to the outside through the window 13 and is consequently emitted as an indication signal 4. In the final result, a user can thus observe the indication light 5, which originates in the interior of the light-guide cable 8, from the outside through the window 13.

In the examples of FIGS. 2 and 3, the infrared illumination light 2 is converted by the converter 3, more specifically by the conversion material 6 that is applied in the form of a conversion layer 11, into visible indication light 5, which has shorter wavelengths than the original illumination light 2. In other words, a conversion is effected from longer wavelengths to shorter wavelengths using the converter 3. If, on the other hand, illumination light 2 that lies in the shortwave UV range is used in a visualization or illumination system 1 according to the invention, the conversion must be effected from shorter wavelengths to longer wavelengths (by the converter 3) so that indication light 5 that is perceivable/visible to humans is produced.

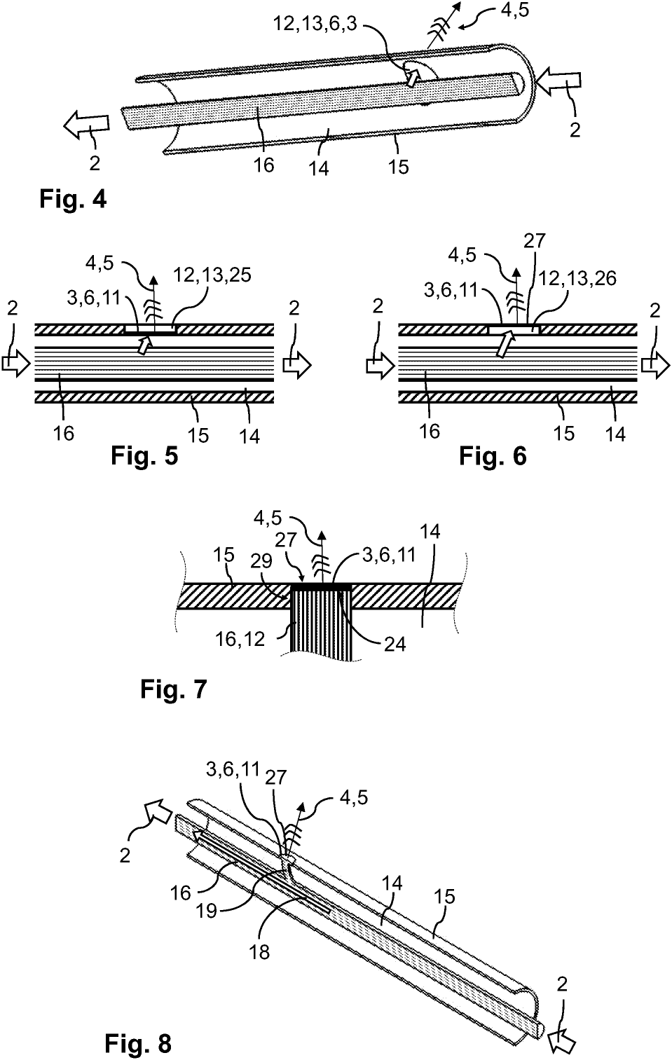

FIG. 4 shows a detail of a further visualization system 1 according to the invention, which could be designed for example as a microscope. This system 1 has a housing 15 which closes an interior space 14. Arranged in the interior 14 is a light guide 16, which (in FIG. 4) transports non-visible illumination light 2 from the right to the left. In this example, too, a converter 3 according to the invention is implemented with the aid of a window 13 that has been placed in a sealing manner into the housing 15. Again, a conversion material 6, which is photoactive and can convert the illumination light 2 into visible indication light 5, is applied on the housing 15, more specifically on the inside on the window 13. As is indicated by the smaller block arrow, the illumination light 2 thus first passes from the light guide 16 onto the inner side of the window 13, is converted there by the conversion material 6 into the indication light 5, and subsequently the indication light 5 passes through the window 13 to the outside.

In order to increase the safety for the user, it is possible in such an innerside arrangement of the conversion material 6 for a filter layer to be applied (on the inside or outside) on the window 13 as well, wherein this filter layer precisely does not transmit the illumination light 2, whereas the filter layer does allow the visible indication light 5 to pass through. It should be understood that, for correct functioning of the converter 3, the conversion material 6 must form the outermost layer (viewed from the inside) so that the illumination light 2 is incident first on the conversion material 6 and only afterwards on the filter layer. This is because in this case the filter layer can block that portion of the non-visible (and potentially dangerous) illumination light 2 that was not absorbed by the conversion material 6.

FIGS. 5 and 6 again explain how a converter 3 according to the invention with a conversion layer 11 located on the inside (FIG. 5) or a conversion layer 11 arranged on the outside (FIG. 6) can be configured: in both examples of FIGS. 5 and 6, a light guide 16 is again arranged in a hermetically sealed interior space 14 of the system 1, wherein by appropriate processing of the light guide 16 at the location of the respectively illustrated window 13, some of the illumination light 2 is branched off such that it is incident on the window 13. In the example of FIG. 5, the conversion layer 11 has been applied on the inside, and so the window 13 there acts as an exit window 25. This is because the visible light 5 is produced in the interior space 14 (by way of the conversion layer 11) and is subsequently transmitted through the window 13 to the outside.

In the example of FIG. 6, by contrast, the window 13 is configured such that it transmits the non-visible illumination light 2. Only after the illumination light 2 has passed through the window 13 does it arrive thus on the conversion layer 11, which has been applied on the outside and again converts the illumination light 2 into visible indication light 5. To protect the conversion layer 11 against mechanical abrasion, it is coated on the outside with a protective layer 27 (which is transparent to the indication light 5). In the example of FIG. 6, the window 13 thus serves as a transmission window 26 for the illumination light 2.

FIG. 7 shows a further possible configuration of a converter 3 according to the invention in a visualization system 1. This system 1 has a light guide 16 which transports non-visible illumination light 2. As is shown in FIG. 8, the light guide 16 has a branch 19 which branches off some of the illumination light 2 from the optical main path 18, which is used for the illumination, and guides it in a short light-guide piece to the housing 15 illustrated in section in FIG. 7. The branched-off piece of the light guide 16 is here, as illustrated in FIG. 7, glued into a through-passage 29 formed in the housing 15 such that the interior space 14 remains sealed off. An end face 24 of the branched-off light-guide piece 16, which serves as a carrier body 12 here, is polished and configured to be planar with the outside of the housing 15. A photoactive conversion layer 11, which implements a converter 3 according to the invention, is applied on this planar light-guide end face 24. In other words, the non-visible illumination light 2 transported by the light guide 16 is again absorbed by the conversion layer 11 and then re-emitted (at least in part in transmission) in the form of the visible indication light 5. In such a configuration, too, an additional protective layer 27 can be applied on the outside to protect the conversion layer 11 against mechanical abrasion, with this protective layer ideally transmitting the indication light 5 but not the non-visible illumination light 2. In this way, the protective layer 27 can after all also increase the safety for the user against the illumination light.

In summary, it is proposed for improving the use properties of a visualization and/or illumination system 1 with which an object can be illuminated with non-visible illumination light 2 during a medical intervention, that some of the illumination light 2 transported by the system 1 is branched off and guided to a converter 3, which picks up, in particular detects or absorbs, the illumination light 2 and subsequently converts it into an indication signal 4 that is perceivable by humans and outputs this indication signal 4. Using the indication signal 4, a user can thus quickly and easily check whether or not the illumination light 2 is currently being emitted by the system 1 (cf. FIG. 1).

List of Reference Signs

| | |
|---|---|
| 1 | Visualization system and/or illumination system for medical applications (for example designed as an endoscope, an exoscope, a microscope or as an illumination optical unit) |
| 2 | Non-visible illumination light |
| 3 | Converter |
| 4 | Indication signal |
| 5 | Indication light |
| 6 | Conversion material |
| 7 | Endoscope |
| 8 | Light-guide cable |
| 9 | Light-guide connection |
| 10 | Light source |
| 11 | Conversion layer (formed from 6) |
| 12 | Carrier body |
| 13 | Window |
| 14 | Interior space (of 1) |
| 15 | Housing |
| 16 | Light guide |
| 17 | Distal end region (e.g. of 7) |
| 18 | Main path (for 2) |
| 19 | Branch |
| 20 | Termination bushing (of 8; can serve, for example, for connecting 8 to 7) |
| 21 | Eyepiece |
| 22 | Endoscope shaft |
| 23 | Distal exit window |
| 24 | End face (of 16) |
| 25 | Exit window (through which 5 radiates) |
| 26 | Transmission window (through which 2 radiates) |
| 27 | Protective layer (transparent to 5) |
| 28 | Cladding |
| 29 | Through-passage |
| 30 | Proximal end (of 7) |

1. A visualization and/or illumination system (1) configured for illuminating an object to be observed during a medical intervention with non-visible illumination light (2) in a first wavelength range, the system comprising:
a converter (3) which converts the non-visible illumination light (2) into an indication signal (4) that is perceivable by humans.

2. The system (1) according to claim 1, wherein the converter (3) outputs the indication signal (4) only if the non-visible illumination light (2) is present.

3. The system (1) according to claim 1, wherein the converter (3) converts at least some of the illumination light (2) into visible indication light (5).

4. The system (1) according to claim 3, wherein the converter is configured to convert the at least some of the illumination light (2) into the visible indication light (5) based on a photoactive substance which enables optical wavelength conversion of the illumination light (2) into the indication light (5) without an external energy supply.

5. The system (1) according to claim 1, wherein the converter (3) comprises a photoactive conversion material (6), and the conversion material (6) is at least one of applied as a conversion layer (11) on a carrier body (12) or embedded in a transparent carrier body (12).

6. The system (1) according to claim 5, wherein the conversion material (6) contains a photoactive substance that is at least one of produced via a sintering method or applied on a ceramic as the carrier body (12).

7. The system (1) according to claim 1, wherein the illumination light (2) is at least one of reflected or transmitted by the converter (3).

8. The system (1) according to claim 1, wherein the converter (3), upon the conversion of the non-visible illumination light (2) into the visible indication light (5), is configured to effect conversion from longer wavelengths to shorter wavelengths.

9. The system (1) according to claim 1, wherein the converter (3), upon the conversion of the non-visible illumination light (2) into the visible indication light (5), is configured to effect conversion from shorter wavelengths to longer wavelengths.

10. The system (1) according to claim 3, wherein the converter (3) is arranged in an interior space (14) of the system (1) and the indication light (5) is at least one of observable through a window (13) from outside the interior space, or applied on an outside of the window (13) or embedded in the window (13), and the non-visible illumination light (2) passes through the window (13) before becoming incident on the conversion material (6).

11. The system (1) according to claim 1, wherein the converter (3) is arranged in or on at least one of a) a housing (15) that surrounds a light guide (16), which propagates the invisible illumination light (2) to an illumination optical unit (16), b) a lightguide cable (8), which supplies the invisible illumination light (2) to an illumination optical unit (16), or c) a catheter configured to introduce the invisible illumination light (2) into a body cavity.

12. The system of claim 11, wherein the converter (3) is arranged such that the converter (3) is located extracorporeally when the visualization and/or illumination system (1) is used.

12. The system (1) according claim 1, further comprising a light source (10), configured to emit the illumination light (2), is arranged in an interior space (14), and a light guide (16) arranged in the interior space (14) configured to guide the illumination light (2) to the converter (3).

13. The system of claim 12, wherein the system comprises an endoscope, and the light source (10) is arranged in a distal end region (17) of the endoscope (7) and the light guide (16) propagates the illumination light (2) in a direction of a proximal end (30) of the endoscope (7).

14. The system (1) according to claim 1, further comprising an internal light guide (16) forming a branch (19) which branches off some of the illumination light (2) from an optical main path (18) used for the illumination and the branch (19) guides some of the illumination light (2) to the converter (3).

15. The system (1) of claim 1, wherein the system comprises an endoscope (7), an exoscope, a microscope, or an illumination optical unit.

16. A method for indicating a presence of non-visible illumination light (2), which is emitted by a visualization and/or illumination system (1) in order to illuminate an object to be observed during a medical intervention, the method comprising:
providing the non-visible illumination light (2), and
converting the non-visible illumination light (2) by a converter (3) into an indication signal (4) that is perceivable by humans, and
outputting the indication signal.

17. The method according to claim 16, further comprising the converter (3) picking up some of the illumination light (2) and, in reaction thereto,
outputting at least one of a) the optical indication signal (4), b) an acoustic indication signal, or c) a mechanical indication signal.

18. The method according to claim 16, further comprising the converter (3) carrying out an optical wavelength conversion of the illumination light (2) into the indication light (5) without an external energy supply.
